(19) United States
(12) Patent Application Publication
(10) Pub. No.: US 2021/0198634 A1
(43) Pub. Date: Jul. 1, 2021

(54) METHODS OF CULTURING PODOCYTES AND COMPOSITIONS THEREOF

(71) Applicant: Beth Israel Deaconess Medical Center, Inc., Boston, MA (US)

(72) Inventors: George C. TSOKOS, Boston, MA (US); Abhigyan SATYAM, Boston, MA (US)

(21) Appl. No.: 17/250,791

(22) PCT Filed: Aug. 30, 2019
(86) PCT No.: PCT/US19/48977

§ 371 Date: Mar. 4, 2021

Related U.S. Application Data
(60) Provisional application No. 62/726,573, filed on Sep. 4, 2018.

(51) Int. Cl. C12N 5/071; A61K 35/22; G01N 33/50

(52) U.S. Cl. CPC C12N 5/0686; A61K 35/22; G01N 33/5014; G01N 2800/245; G01N 2800/347; G01N 2800/50; C12N 2533/90

(57) ABSTRACT

Provided herein are methods of growing podocytes in culture. Also provided are cell culture systems comprising decellularized extracellular matrix, tissue culture substrates, and podocytes. Further provided are podocytes produced by the methods and cell culture systems described herein and methods of using the podocytes.

### CROSS REFERENCE TO RELATED APPLICATIONS

This application claims priority to U.S. Provisional Patent Application No. 62/726,573, filed Sep. 4, 2018, the disclosure of which is incorporated herein by reference in its entirety.

### STATEMENT REGARDING FEDERALLY FUNDED RESEARCH

This invention was made with government support under Grant No. AR064350 awarded by the Nation Institutes of Health. The government has certain rights in the invention.

### FIELD OF THE INVENTION

The present invention is directed generally to the field of biotechnology. More particularly to the field of cell culture and tissue regeneration. Provided are methods and cell culture systems for enhancing the survival and differentiation of podocytes. Also provided are podocytes that are grown and differentiated utilizing the methods and cell culture systems described herein.

### BACKGROUND OF THE INVENTION

The study of renal diseases are intricate because of hidden onset, the genetic makeup of host, and/or the acute or chronic nature of the disease. Most investigators rely on humans subjects as a gold standard to study disease susceptibility, disease mechanism(s), disease prognosis, and potential disease therapies. However, many kidney diseases are not that common in the general population, which increases the challenge to find alternative disease models. With the development of a gene editing technique, the use of experimental animal models have proven to be instrumental and has significantly advanced the understanding of many aspects of kidney disease. Nevertheless, animal models do not always fully replicate their human counterpart and therefore the use of human cells grown in vitro becomes a necessity (Shankland et al., Kidney International 72(1):26-36 (2007)).

One of the fastest moving areas of research progress in nephrology has been the appreciation of the importance of the glomerular epithelial cell, known as the podocyte (Mathieson, Current Opinion Nephrology Hypertension 18(3):206-11 (2009); Patrakka et al., Nephrology 5(8):463-8 (2009)). Podocytes are the pivotal cells maintaining normal structure and function of the kidney glomerulus (Kriz et al., Kidney Int. 54(3):687-97 (1998); Pavenstadt et al., Physiological Reviews 83(1):253-307 (2003)). Podocytes play a key role in the prevention of proteinuria, and these cells are important targets of injury in a variety of renal diseases and are important determinants of outcome (Mathieson, Current Opinion Nephrology Hypertension 18(3):206-11 (2009); Patrakka et al., Nephrology 5(8):463-8 (2009)). Loss of podocytes is associated with progression of kidney disease in humans and experimental animals (Pagtalunan et al., J. Clin. Invest. 99(2):342-8 (1997); Fukuda et al., Kidney Int. 81(1):40-55 (2012)) since there is at most a limited possibility to replace these post-mitotic terminally differentiated cells. Moreover, attachment of podocyte foot processes to the glomerular basement membrane (GBM) makes direct isolation of podocytes difficult, so in vitro studies of these cells depend largely on cell culture systems (Da Sacco et al., PLOS ONE 8(12):e81812 (2013)).

In vitro study of podocytes used to be difficult because of lack of techniques for obtaining differentiated cells in quantities adequate for research. Recently, conditionally immortalized human podocyte cell lines have been developed by transfecting them using both the temperature-sensitive mutant U19tsA58 of the SV40 large T antigen (SV40) and the essential catalytic subunit of the hTERT telomerase gene (O'Hare et al., PNAS 98(2):646-51 (2001); Saleem et al., J. Amer. Soc. Nephrology 13(3):630-8 (2002)). Transfection of cells with SV4OT allows cells to proliferate at the ‘permissive’ temperature of 33° C. Transfer to the ‘non-permissive’ temperature of 37° C. results in the inactivation of large T antigen with minor changes in gene expression (Stamps et al., Int. J. Canc. 57(6):865-74 (1994)). Podocytes then enter growth arrest and express markers of differentiated in vivo podocytes, including the novel podocyte proteins, nephrin, podocin, CD2AP, and synaptopodin, and known molecules of the slit diaphragm ZO-1; alpha-, beta-, and gamma-catenin; and P-cadherin (Saleem et al., Amer. J. Pathol. 161(4):1459-66 (2002)). These conditionally immortalized human podocyte cell lines have been used to understand the biology of podocytes in vivo (Shankland et al., Kidney Int. 72(1):26-36 (2007)). However, there are evident differences in the morphology and gene expression between cultured podocytes and their in vivo counterparts (Chittiprol et al., Amer. J. Physiol. Renal Physiol. 301(3):F660-71 (2011)). Podocytes in vivo project elaborate cell processes referred to as primary processes and foot processes (Ichimura et al., Scientific Reports 5:8993 (2015)). These processes interdigitate with each other. Cultured podocytes also form elongate cell processes when there is cell-free space around cells (Mundel et al., Experimental Cell Res. 236(1):248-58 (1997); Kobayashi et al., Italian J. Anat. Embryology 106(2 Suppl. 1):423-30 (2001); Gao et al., Nephron Experimental Nephrology 97(2):e49-61 (2004)). However, they lose such cell processes when they reach a confluent density similar to the cell density in vivo (Chittipol et al., Amer. J. Physiol. Renal Physiol. 301(3):F660-71 (2011)). Despite efficacious in vitro and in vivo results from various techniques, the culture of podocytes is still a challenge, primarily due to seeding cells directly on tissue culture plastic impedes the native podocyte function in vitro. The current podocyte based technologies face challenges of expanding cell numbers and directing differentiation while maintaining native phenotype, physiology, and therapeutic potential. In the field of kidney research there is an urgent need to generate physiological and pathophysiological cell systems and tissue-like constructs for diagnostics, disease models and drug or gene screening purposes.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE INVENTION

In one general aspect, the invention relates to methods of growing podocytes in culture. The methods comprise (a) contacting a tissue culture substrate with cells; (b) growing the cells on the tissue culture substrate; (c) inducing the cells to produce an extracellular matrix (ECM); (d) decellularizing the ECM to produce a decellularized ECM; and (e) contacting the decellularized ECM with podocytes under conditions suitable to grow the podocytes, wherein the podocytes are grown in culture. In certain embodiments, the cells are selected from the group consisting of fibroblasts, stem cells, glomerular parietal epithelial cells, renal pericytes, mesangial cells, and renal tubule epithelial cells.

In certain embodiments, the tissue culture substrate is selected from the group consisting of a tissue culture plastic, a glass container, a bioceramic container, a stainless steel container, and a polymeric container.

In certain embodiments, inducing the cells to produce extracellular matrix comprises contacting the cells with an induction agent capable of inducing collagen production from the cells. The induction agent can, for example, be selected from the group consisting of ascorbic acid phosphate, polysaccharide, hyaluronic acid, polyethylene glycol, and polyvinylpyrrolidone.

In certain embodiments, decellularizing the ECM to produce a decellularized extracellular matrix comprises contacting the cells and ECM with a decellularizing agent capable of reducing or eliminating cellular components from the ECM. The decellularizing agent can, for example, be selected from the group consisting of a mild detergent, an enzyme, double distilled water, acids, bases, and mechanical decellularization.

In certain embodiments, the mild detergent is selected from the group consisting of sodium deoxycholate, Triton X-100, and sodium dodecyl sulfate (SDS).

In certain embodiments, the methods provided herein further comprise differentiating the podocytes grown in culture.

Also provided are podocyte cells produced by the methods of the invention.

Also provided are pharmaceutical compositions comprising a podocyte cell produced by the methods of the invention and a pharmaceutically acceptable carrier.

Also provided are podocyte cell culture systems. The podocyte cell culture system comprises (a) a decellularized extracellular matrix (ECM); (b) a tissue culture substrate; and (c) a podocyte. In certain embodiments, the tissue culture substrate is selected from the group consisting of a tissue culture plate, a glass container, a bioceramic container, a stainless steel container, and a polymeric container. In certain embodiments, the decellularized ECM is produced by (a) growing cells on a tissue culture substrate; (b) inducing the cells to produce an extracellular matrix (ECM); and (c) decellularizing the ECM to produce a decellularized ECM.

Also provided are methods of determining if a subject with lupus is at risk of developing glomerulonephritis. The methods comprise (a) obtaining sera from a subject with lupus; (b) contacting the sera with the podocyte cell culture system of the invention; and (c) detecting damage to the podocyte cell culture, wherein detecting damage to the podocyte cell culture indicates that the subject is at risk of developing glomerulonephritis.

Also provided are methods of determining if a subject with a kidney transplant is at risk of rejecting the kidney transplant. The methods comprise (a) obtaining sera from a subject with a kidney transplant; (b) contacting the sera with the podocyte cell culture system of the invention; and (c) detecting damage to the podocyte cell culture, wherein detecting damage to the podocyte cell culture indicates that the subject is at risk of rejecting the kidney transplant. In certain embodiments, the method further comprises administering a therapeutic to the subject to reduce the risk of rejecting the kidney transplant. The therapeutic can, for example, be a pharmaceutical composition comprising a podocyte cell and a pharmaceutically acceptable carrier, the podocyte cell produced by a method of invention provided herein.

Also provided are methods of determining if a kidney is at risk to cytotoxicity due to a therapeutic agent. The methods comprise (a) contacting the therapeutic agent with the podocyte cell culture system of the invention; and (b) detecting damage to the podocyte cell culture, wherein detecting damage to the podocyte cell culture indicates that the kidney is at risk to cytotoxicity due to the therapeutic agent. In certain embodiments, the therapeutic agent is selected from an antibiotic, a chemotherapeutic agent, a therapeutic peptide, and a therapeutic small molecule.

Further aspects, features and advantages of the present invention will be better appreciated upon a reading of the following detailed description of the invention and claims.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1B show different podocyte culturing systems. In a conventional podocyte culture system, the morphology and physiology of cells are far from physiological conditions (FIG. 1A). Podocytes cultured on decellularized matrix can help to interdigitate foot processes and imitate the in vivo physiology (FIG. 1B). The decellularized matrix contains a dense array of extracellular matrix deposited by human fibroblasts.

FIG. 2 shows immunofluorescence analysis of decellularized matrix confirmed the deposition of collagen type I, IV, and fibronectin.

FIGS. 3A-3C show graphs of AlamarBlue® assays, which demonstrate that decellularized matrix helped to increase viability of podocytes.

FIG. 4 shows immunofluorescence analysis that demonstrates differentiation of human podocytes on plastic or decellularized fibroblast ECM rich substrate. Phalloidin staining was performed for actin filaments.

FIGS. 5A-5B show immunofluorescence analysis demonstrating podocytes cultured on decellularized ECM rich substrate showed visibly higher expression of synaptopodin (FIG. 5A) and nephrin (FIG. 5B) up to 21 days under differentiation conditions.

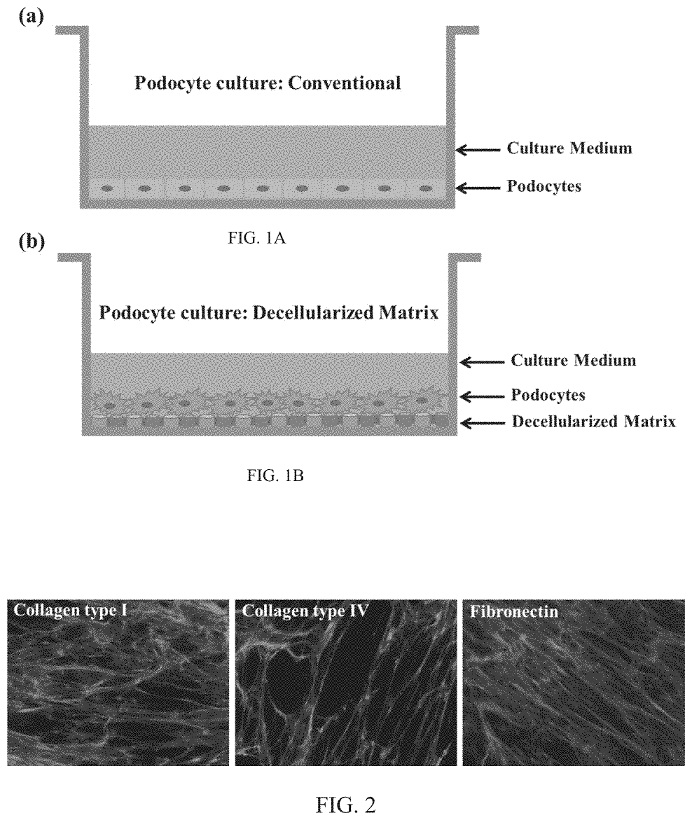

### DETAILED DESCRIPTION OF THE INVENTION

This invention is based, in part, on the identification of a cell culture system for enhancing the survival and differentiation of podocytes grown in culture. Current podocyte based technologies face challenges of expanding cell numbers and directing differentiation while maintaining native phenotype, physiology, and therapeutic potential. Provided herein are methods for growing and differentiating podocyte cells in cell culture systems. These cell culture systems utilize a biophysical approach termed macromolecular crowding (MMC), as a means to create extracellular matrix (ECM)-rich tissue equivalents and decellularization to remove intracellular milieu. The decellularized ECM serve as a scaffold for new cells (e.g., podocytes) to grow in an environment similar to native conditions, as decellularization minimally affects the tissue microstructure. The preservation of this microstructure and other functional components is believed to enhance cell attachment and proliferation, which can be used for the healing of damaged tissue. The cell culture systems provided herein provide better microenvironments for podocytes to proliferate and differentiate while maintaining the native phenotype, physiology, and therapeutic potential.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, “subject” or “patient” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

As used herein, “sample” is intended to include any sampling of cells, tissues, or bodily fluids in which expression of a biomarker can be detected. Examples of such samples include, but are not limited to, biopsies, smears, blood, lymph, urine, saliva, or any other bodily secretion or derivative thereof. Blood can, for example, include whole blood, plasma, serum, or any derivative of blood. Samples can be obtained from a subject by a variety of techniques, which are known to those skilled in the art.

As used herein, the terms “extracellular matrix” and “ECM” refer to a natural or artificial scaffolding for cell growth. Natural ECMs (ECMs found in multicellular organisms, such as mammals and humans) are complex mixtures of structural and non-structural biomolecules, which can include, but are not limited to, collagens, elastins, laminins, glycosaminoglycans, proteoglycans, antimicrobials, chemoattractants, cytokines, and growth factors. In mammals, ECM often comprises about 90% collagen, in its various forms. The composition and structure of ECMs vary depending on the source of the tissue. For example, small intestine submucosa (SIS), urinary bladder matrix (UBM) and liver stroma ECM each differ in their overall structure and composition due to the unique cellular niche needed for each tissue.

As used herein, the term “decellularized” refers to the removal of cells and their related debris, for example, from the ECM. Removal of cells and their related debris from ECM produces a decellularized ECM (DM).

Methods of Growing and Differentiating Podocytes

In one general aspect, provided herein are methods of growing podocytes in culture. The methods comprise (a) contacting a tissue culture substrate with cells; (b) growing the cells on the tissue culture substrate; (c) inducing the cells to produce an extracellular matrix (ECM); (d) decellularizing the ECM to produce a decellularized ECM; and (e) contacting the decellularized ECM with podocytes under conditions suitable to grow the podocytes, wherein the podocytes are grown in culture. In certain embodiments, the cells are selected from the group consisting of fibroblasts, stem cells, glomerular parietal epithelial cells, renal pericytes, mesangial cells, and renal tubule epithelial cells.

In certain embodiments, the tissue culture substrate is selected from the group consisting of a tissue culture plastic, a glass container, a bioceramic container, a stainless steel container, and a polymeric container. The tissue culture substrate can be coated with a sheet of electrospun biodegradable polymers. The biodegradably polymers can, for example, be selected from poly(ε-caprolactone), poly(Lactic-co-glycolic acid), poly(L-lactide), and polyethylene glycol.

In certain embodiments, inducing the cells to produce extracellular matrix comprises contacting the cells with an induction agent capable of inducing collagen production from the cells. The induction agent can, for example, be selected from the group consisting of ascorbic acid phosphate, polysaccharide, hyaluronic acid, polyethylene glycol, and polyvinylpyrrolidone.

Removal of the cells can be performed by any method useful for decellularization while retaining a decellularized ECM. In certain embodiments, decellularizing the ECM to produce a decellularized ECM comprises contacting the cells and ECM with a decellularizing agent capable of reducing or eliminating cellular components from the ECM. The decellularizing agent can, for example, be selected from the group consisting of a mild detergent, an enzyme, double distilled water, acids, bases, and mechanical decellularization.

Removal methods can include, for example, treatment with a detergent. The detergent can, for example, be selected from the group consisting of sodium deoxycholate, Triton X-100, CHAPS, and sodium dodecyl sulfate (SDS). In certain embodiments, decellularizing the ECM can, for example, include treatment with one or more of detergents. The detergents can solubilize the cell membranes and fat to aid in the removal of the cellular debris from the ECM. Residual detergent can be eliminated after the decellularization process by methods known in the art, such that the decellularized ECM can be used in the methods of culturing podocytes described herein.

Removal methods can include, for example, mechanical decellularization. Mechanical decellularization can, for example, include the use of high hydrostatic pressure or a freeze-thaw (e.g., a supercritical carbon dioxide freeze-thaw).

Removal methods can include, for example, treatment with double distilled water, acids (e.g., peracetic acid, oxalic acid, ethylenediaminetetraacetic acid), and/or bases (e.g., reversible alkaline swelling, sodium hydroxide (NaOH)). These solutions can generally be referred to as hypotonic solutions, which serve to lyse the cells in the ECM. In certain aspects, the hypotonic solutions can comprise protease inhibitors to counteract proteases released by the cell during the process of cell lysis. Examples of protease inhibitors include, but are not limited to, 4-(-2-aminoethyl)-benzene-sulfonyl fluoride, E-64, bestatin, leopeptin, aprotin, PMSF, Na EDTA, TIMPS, pepstatin A, phosphoramidon, and 1,10-phenanthroline.

Removal methods can include, for example, treatment with enzymes. The enzymes can include, but are not limited to, nucleases (e.g., DNase, exonucleases, endonucleases) and phospholipases (e.g., phospholipase A or C). Generally, the cells are treated with nuclease to remove DNA and RNA. Nucleases can inhibit the cellular metabolism, protein production, and cell division of a cell without degrading the collagen matrix. Phospholipases can inhibit cellular function by disrupting cellular membranes. The enzymes provided to the ECM can be provided in buffer solutions or hypotonic solutions. The ionic concentration, the pH, the treatment temperature, and the length of treatment can be optimized to ensure the proper decelullarization of the ECM without affecting the residual collagen matrix.

In certain embodiments, the methods provided herein further comprise differentiating the podocytes grown in culture. The cell culture systems and methods provided herein, that is, the use of decellularized ECM on tissue culture substrates, allow for the enhanced survival, growth, and differentiation as compared to non-coated tissue culture substrates. The use of the cell culture systems and methods of the invention allow for the podocytes to exhibit native morphology with interdigitating foot processes under conditions for differentiation.

Also provided are podocyte cells produced by the methods of the invention.

Pharmaceutical Compositions

In another general aspect, the invention relates to pharmaceutical compositions comprising podocyte cells produced by the methods of the invention and a pharmaceutically acceptable carrier. Podocyte cells produced by the methods of the invention and compositions comprising them are also useful in the manufacture of a medicament for therapeutic applications mentioned herein.

By “pharmaceutical composition” is meant any composition that contains a therapeutically or biologically active agent, such as population of cells for tissue regeneration or therapeutic treatment (e.g., a podocyte cell produced by the methods of the invention) that is suitable for administration to a subject and that treats or prevents a renal disease (e.g., glomerulonephritis, focal segmental glomerulosclerosis, diabetic nephropathy, membranous nephropathy, lupus nephritis) or reduces or ameliorates one or more symptoms of the disease. For the purposes of this invention, pharmaceutical compositions include pharmaceutical compositions suitable for delivering the therapeutic or biologically active agent can include, for example, tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{st} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a pharmaceutical composition can be used in the invention.

Pharmaceutically acceptable acidic/anionic salts for use in the invention include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide. Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol (also known as tris(hydroxymethyl)aminomethane, tromethane or “TRIS”), ammonia, benzathine, t-butylamine, calcium, chloroprocaine, choline, cyclohexylamine, diethanolamine, ethylenediamine, lithium, L-lysine, magnesium, meglumine, N-methyl-D-glucamine, piperidine, potassium, procaine, quinine, sodium, triethanolamine, or zinc.

The pharmaceutical composition can have a pH from about 3.0 to about 10, for example from about 3 to about 7, or from about 5 to about 9. The formulation can further comprise at least one ingredient selected from the group consisting of a buffer system, preservative(s), tonicity agent(s), chelating agent(s), stabilizer(s) and surfactant(s).

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carriers can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via a syringe or an infusion pump. The injection can be delivered subcutaneously, intramuscularly, intraperitoneally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of the embodiment include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars can be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA (ethylenediamine tetraacetic acid), and/or benzamidine hydrochloric acid (HCl). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

The pharmaceutical composition of the invention can comprise an amount of an amino acid base sufficient to decrease aggregate formation of the polypeptide during storage of the composition. The term “amino acid base” refers to one or more amino acids (such as methionine, histidine, imidazole, arginine, lysine, isoleucine, aspartic acid, tryptophan, threonine), or analogues thereof. Any amino acid can be present either in its free base form or in its salt form. Any stereoisomer (i.e., L, D, or a mixture thereof) of the amino acid base can be present. The amino acid base can be present individually or in the combination with other amino acid bases, in a concentration from about 0.01 mg/ml to about 50 mg/ml, for example from about 0.1 mg/ml to about 20 mg/ml. Pharmaceutical compositions comprising each one of these specific amino acid bases constitute alternative embodiments of the invention.

It is also apparent to one skilled in the art that the therapeutically effective dose for podocytes produced by methods and cell culture systems of the invention or a pharmaceutical composition thereof will vary according to the desired effect. Therefore, optimal dosages to be administered can be readily determined by one skilled in the art and will vary with the particular podocytes used, the mode of administration, the strength of the preparation, and the advancement of the disease condition (e.g., glomerulonephritis, focal segmental glomerulosclerosis, diabetic nephropathy, membranous nephropathy, lupus nephritis). In addition, factors associated with the particular subject being treated, including subject age, weight, diet and time of administration, will result in the need to adjust the dose to an appropriate therapeutic level.

The pharmaceutically-acceptable salts of the adenoviral particles of the invention include the conventional non-toxic salts or the quaternary ammonium salts which are formed from inorganic or organic acids or bases. Examples of such acid addition salts include acetate, adipate, benzoate, benzenesulfonate, citrate, camphorate, dodecylsulfate, hydrochloride, hydrobromide, lactate, maleate, methanesulfonate, nitrate, oxalate, pivalate, propionate, succinate, sulfate and tartrate. Base salts include ammonium salts, alkali metal salts such as sodium and potassium salts, alkaline earth metal salts such as calcium and magnesium salts, salts with organic bases such as dicyclohexylamino salts and salts with amino acids such as arginine. Also, the basic nitrogen-containing groups can be quaternized with, for example, alkyl halides.

The pharmaceutical compositions of the invention can be administered by any means that accomplish their intended purpose. As used herein, by “administering” is meant a method of giving a dosage of a pharmaceutical composition (e.g., a podocyte produced by the methods and cell culture systems of the invention) to a subject. The compositions utilized in the methods described herein can be administered, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the severity of the condition being treated).

Podocyte Cell Culture Systems

In another general aspect of the invention, provided are podocyte cell culture systems. The podocyte cell culture system comprises (a) a decellularized extracellular matrix (ECM); (b) a tissue culture substrate; and (c) a podocyte. In certain embodiments, the tissue culture substrate is selected from the group consisting of a tissue culture plate, a glass container, a bioceramic container, a stainless steel container, and a polymeric container. In certain embodiments, the decellularized ECM is produced by (a) growing cells on a tissue culture substrate; (b) inducing the cells to produce an extracellular matrix (ECM); and (c) decellularizing the ECM to produce a decellularized ECM.

Methods of Use

The present invention provides methods for treatment or prevention of a kidney disease (e.g., glomerulonephritis, focal segmental glomerulosclerosis, diabetic nephropathy, membranous nephropathy, lupus nephritis). The methods comprise administering to the subject a pharmaceutical composition comprising podocytes produced by the methods and cell culture systems of the invention. The methods are for treating, preventing, or delaying the onset of, or ameliorating the kidney disease or any one or more symptoms of the kidney disease, the methods comprising administering to the subject in need thereof an effective amount of a pharmaceutical composition of the invention.

According to particular embodiments, an effective or protective amount refers to the amount of the pharmaceutical composition which is sufficient to achieve one, two, three, four, or more of the following effects: (i) reduce or ameliorate the severity of the kidney disease to be treated or a symptom associated therewith; (ii) reduce the duration of the kidney disease to be treated, or a symptom associated therewith; (iii) prevent the progression of the kidney disease to be treated, or a symptom associated therewith; (iv) cause regression of the kidney disease to be treated, or a symptom associated therewith; (v) prevent the development or onset of the kidney disease to be treated, or a symptom associated therewith; (vi) prevent the recurrence of the kidney disease to be treated, or a symptom associated therewith; (vii) reduce hospitalization of a subject having the kidney disease to be treated, or a symptom associated therewith; (viii) reduce hospitalization length of a subject having the kidney disease to be treated, or a symptom associated therewith; (ix) increase the survival of a subject with the kidney disease to be treated, or a symptom associated therewith; (xi) inhibit or reduce the kidney disease to be treated, or a symptom associated therewith in a subject; and/or (xii) enhance or improve the prophylactic or therapeutic effect(s) of another therapy.

The effective amount or dosage can vary according to various factors, such as the kidney disease to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

As used herein, the terms “treat,” “treating,” and “treatment” are all intended to refer to an amelioration or reversal of at least one measurable physical parameter related to the kidney disease, which is not necessarily discernible in the subject, but can be discernible in the subject. The terms “treat,” “treating,” and “treatment,” can also refer to causing regression, preventing the progression, or at least slowing down the progression of the kidney disease. In a particular embodiment, “treat,” “treating,” and “treatment” refer to an alleviation, prevention of the development or onset, or reduction in the duration of one or more symptoms associated with the kidney disease. In a particular embodiment, “treat,” “treating,” and “treatment” refer to prevention of the recurrence of the kidney disease. In a particular embodiment, “treat,” “treating,” and “treatment” refer to an increase in the survival of a subject having the kidney disease. In a particular embodiment, “treat,” “treating,” and “treatment” refer to elimination of the kidney disease in the subject.

According to particular embodiments, also provided are methods of determining if a subject with lupus is at risk of developing glomerulonephritis. The methods comprise (a) obtaining sera from a subject with lupus; (b) contacting the sera with the podocyte cell culture system of the invention; and (c) detecting damage to the podocyte cell culture, wherein detecting damage to the podocyte cell culture indicates that the subject is at risk of developing glomerulonephritis.

According to particular embodiments, also provided are methods of determining if a subject with a kidney transplant is at risk of rejecting the kidney transplant. The methods comprise (a) obtaining sera from a subject with a kidney transplant; (b) contacting the sera with the podocyte cell culture system of the invention; and (c) detecting damage to the podocyte cell culture, wherein detecting damage to the podocyte cell culture indicates that the subject is at risk of rejecting the kidney transplant. In certain embodiments, the method further comprises administering a therapeutic to the subject to reduce the risk of rejecting the kidney transplant. The therapeutic can, for example, be a pharmaceutical composition comprising a podocyte cell and a pharmaceutically acceptable carrier, the podocyte cell produced by a method of invention provided herein.

According to particular embodiments, also provided are methods of determining if a kidney is at risk to cytotoxicity due to a therapeutic agent. The methods comprise (a) contacting the therapeutic agent with the podocyte cell culture system of the invention; and (b) detecting damage to the podocyte cell culture, wherein detecting damage to the podocyte cell culture indicates that the kidney is at risk to cytotoxicity due to the therapeutic agent. In certain embodiments, the therapeutic agent is selected from an antibiotic, a chemotherapeutic agent, a therapeutic peptide, and a therapeutic small molecule.

### EMBODIMENTS

The invention provides also the following non-limiting embodiments.

Embodiment 1 is method of growing podocytes in culture, the method comprising:
a. contacting a tissue culture substrate with cells;
b. growing the cells on the tissue culture substrate;
c. inducing the cells to produce an extracellular matrix (ECM);
d. decellularizing the ECM to produce a decellularized ECM; and
e. contacting the decellularized extracellular matrix with podocytes under conditions suitable to grow the podocytes, wherein the podocytes are grown in culture.

Embodiment 2 is the method of embodiment 1, wherein the cells are selected from the group consisting of fibroblasts, stem cells, glomerular parietal epithelial cells, renal pericytes, mesangial cells, and renal tubule epithelial cells.

Embodiment 3 is the method of embodiment 1 or 2, wherein the tissue culture substrate is selected from the group consisting of a tissue culture plastic, a glass container, a bioceramic container, a stainless steel container, and a polymeric container.

Embodiment 4 is the method of embodiment 3, wherein the tissue culture substrate is a tissue culture plastic.

Embodiment 5 is the method of embodiment 3 or 4, wherein the tissue culture substrate can be coated with an electrospun biodegradable polymer.

Embodiment 6 is the method of embodiment 5, wherein the electrospun biodegradable polymer comprises poly(ε-caprolactone), poly(lactic-co-glycolic acid), poly(L-lactide), or polyethylene glycol.

Embodiment 7 is the method of any one of embodiments 1-6, wherein inducing the cells to produce extracellular matrix comprises contacting the cells with an induction agent capable of inducing collagen production from the cells.

Embodiment 8 is the method of embodiment 7, wherein the induction agent is selected from the group consisting of ascorbic acid phosphate, polysaccharide, hyaluronic acid, polyethylene glycol, and polyvinylpyrrolidone.

Embodiment 9 is the method of any one of embodiments 1-8, wherein decellularizing the ECM to produce a decellularized ECM comprises contacting the cells and ECM with a decellularizing agent capable of reducing or eliminating cellular components from the ECM.

Embodiment 10 is the method of embodiment 9, wherein the decellularizing agent is selected from the group consisting of a mild detergent, an enzyme, double distilled water, acids, bases, and mechanical decellularization.

Embodiment 11 is the method of embodiment 10, wherein the mild detergent is selected from the group consisting of sodium deoxycholate, Triton X-100, CHAPS, and sodium dodecyl sulfate (SDS).

Embodiment 12 is the method of embodiment 10, wherein the mechanical decellularization is selected from high hydrostatic pressure or supercritical carbon dioxide free-thaw.

Embodiment 13 is the method of any one of embodiments 1-12, wherein the method further comprises differentiating the podocytes grown in culture.

Embodiment 14 is a podocyte cell produced by the method of any one of embodiments 1-13.

Embodiment 15 is a pharmaceutical composition comprising a podocyte cell of embodiment 14 and a pharmaceutically acceptable carrier.

Embodiment 16 is a podocyte cell culture system, the podocyte cell culture system comprising:
a. a decellularized extracellular matrix (ECM);
b. a tissue culture substrate; and
c. a podocyte.

Embodiment 17 is the podocyte cell culture system of embodiment 16, wherein the tissue culture substrate is selected from the group consisting of a tissue culture plate, a glass container, a bioceramic container, a stainless steel container, and a polymeric container.

Embodiment 18 is the podocyte cell culture system of embodiment 16, wherein the decellularized ECM is produced by (a) growing cells on a tissue culture substrate; (b) inducing the cells to produce an ECM; and (c) decellularizing the ECM to produce a decellularized ECM.

Embodiment 19 is a method of determining if a subject with lupus is at risk of developing glomerulonephritis, the method comprising:
a. obtaining sera from a subject with lupus;
b. contacting the sera with the podocyte cell culture system of any one of embodiments 16-18; and
c. detecting damage to the podocyte cell culture, wherein detecting damage to the podocyte cell culture indicates that the subject is at risk of developing glomerulonephritis.

Embodiment 20 is a method of determining if a subject with a kidney transplant is at risk of rejecting the kidney transplant, the method comprising:
a. obtaining sera from a subject with a kidney transplant;
b. contacting the sera with the podocyte cell culture system of any one of embodiments 16-18; and
c. detecting damage to the podocyte cell culture, wherein detecting damage to the podocyte cell culture indicates that the subject is at risk of rejecting the kidney transplant.

Embodiment 21 is the method of embodiment 20, wherein the method further comprises administering a therapeutic to the subject to reduce the risk of rejecting the kidney transplant.

Embodiment 22 is the method of embodiment 21, wherein the therapeutic is a pharmaceutical composition comprising a podocyte cell and a pharmaceutically acceptable carrier, the podocyte produced by a method of growing podocytes in culture comprising:
a. contacting a tissue culture substrate with cells;
b. growing the cells on the tissue culture substrate;
c. inducing the cells to produce an extracellular matrix (ECM);
d. decellularizing the ECM to produce a decellularized ECM; and
e. contacting the decellularized ECM with podocytes under conditions suitable to grow the podocytes.

Embodiment 23 is a method of determining if a kidney is at risk to cytotoxicity due to a therapeutic agent, the method comprising:
a. contacting the therapeutic agent with the podocyte cell culture system of any one of embodiments 16-18; and
b. detecting damage to the podocyte cell culture, wherein detecting damage to the podocyte cell culture indicates that the kidney is at risk to cytotoxicity due to the therapeutic agent.

Embodiment 24 is the method of embodiment 23, wherein the therapeutic agent is selected from an antibiotic, a chemotherapeutic agent, a therapeutic peptide, and a therapeutic small molecule.

### EXAMPLES

### Example 1: Decellularized Extracellular Matrix Rich Biomimetic Substrate Supports the Proliferation, Differentiation, and Maintenance of Native Phenotype, Physiology, and Therapeutic Potential for Podocyte Cells

Materials and Methods

Cell culture: Human skin fibroblasts were cultured in Dulbecco's modified Eagle medium (DMEM) with 10% fetal bovine serum (FBS) and 1% penicillin-streptomycin at 37° C. in a humidified atmosphere of 5% CO₂. Fibroblasts were seeded at 25,000 cells/cm² in 24-well or 12-well plates and were allowed to attach for 24 hours. After 24 hours the medium was changed with medium containing 100 μM L-ascorbic acid phosphate to induce collagen synthesis. Human podocytes were cultured in Roswell Park Memorial Institute (RPMI)—1640 medium with 10% fetal bovine serum (FBS), 1% Insulin-transferrin-sodium selenite and 1% penicillin-streptomycin at 33° C. in a humidified atmosphere of 5% CO₂. Podocytes were moved to 37° C. after reaching 80% confluency.

Construction of decellularized matrix: Human skin fibroblasts were cultured for 7 days with 100 μM L-ascorbic acid phosphate supplementation. At the end of culture time, medium was removed and cells were washed twice with PBS. Fibroblasts were decellularized using 0.5% sodium deoxycholate with or without Triton X-100 and EDTA. Decellularized well plates were washed 6 times with phosphate buffer saline (PBS) and sterilized under ultraviolet light for 2 hours.

Phase contrast microscopy: The influence of decellularized matrix on podocyte morphology was evaluated using phase-contrast microscopy at various culture time. Images of the podocytes were taken using a Nikon eclipse 80i microscope and analyzed using Nikon NIS-Elements software (Nikon; Tokyo, Japan).

Cell metabolic activity and viability: alamarBlue® (Invitrogen; Carlsbad, Calif.) cell metabolic activity assay was performed to quantify cell viability of the podocytes. Briefly, at the end of culture time points, cells were washed with Hanks' Balanced Salt solution (HBSS, Sigma, US) and then diluted alamarBlue® was added. After 4 hours of incubation at 37° C., absorbance was measured at 550 and 595 nm with help of Synergy™ HT multi-mode microplate reader (BioTek Instruments; Winooski, Vt.). Cell metabolic activity was expressed in terms of reduction percentage of alamarBlue®.

Quantification of deposited collagen in decellularized matrix: Decellularized fibroblast matrix was digested with porcine gastric mucosa pepsin (Sigma, US) in a final concentration of 1 mg/ml in 0.5M acetic acid (Sigma; St. Louis, Mo.). Samples were incubated at 37° C. for 2 hours with gentle shaking followed by neutralization with 0.1N sodium hydroxide (Sigma). The extracted collagen samples were analyzed by SDS-PAGE (Sodium dodecyl sulphate-polyacrylamide gel electrophoresis) under non-reducing conditions. 100-500 μg/ml of rat tail collagen type I (Sigma) was used as standards with every gel. Protein bands were stained with the coomassie blue or silver stain. Densitometric analysis of gels was performed using ImageJ 1.44 (NIH; Bethesda, Md.) software. Collagen bands were quantified by defining each band with the rectangular tool with background subtraction.

Western Blotting: The total protein from human podocytes was extracted using radio immunoprecipitation assay (RIPA) buffer. Protein concentrations were normalized for all samples and separated on NuPAGE Novex 4-12% Bis-Tris gels (ThermoFisher Scientific; Waltham, Mass.). Subsequently, the proteins were transferred to polyvinylidene difluoride membranes (ThermoFisher Scientific), blocked with 5% skimmed milk in tris-buffered saline tween (TB ST), and incubated overnight with guinea pig anti-nephrin antibody (Progen Biotechnik; Heidelberg, Germany) at a dilution 1:500, goat anti-synaptopodin antibody (Santa Cruz Biotechnology; Dallas, Tex.) at a dilution 1:1000, rabbit anti-wilms' tumor (WT) 1 antibody (Santa Cruz Biotechnology) at a dilution 1:500, mouse anti-glyceraldehyde-3-phosphate dehydrogenase (GAPDH) antibody (BioLegend; San Diego, Calif.) at dilution a 1:5000, and mouse anti-β-actin antibody (Sigma-Aldrich) at dilution a 1:5000 in TBST containing 5% skimmed milk. After three washes with TBST, the membranes were incubated with appropriate horseradish peroxidase-conjugated secondary antibodies (Santa Cruz Biotechnology). Protein bands were visualized using a chemiluminescent ECL™ detection kit (GE Healthcare; Little Chalfont, United Kingdom).

Immunocytochemistry: Human skin fibroblasts were seeded on 4 or 8-well Lab-Tek™ II (Nunc, Denmark) chamber slides at 25,000 cells/cm² and were decellularized. Human podocytes were seeded on decellularized matrix on normal on 4 or 8-well Lab-Tek™ II chamber slides. At the end of cell culture time points, medium was removed and cell layers were washed with HBSS and fixed with 4% paraformaldehyde (Sigma) at room temperature for 15 minutes. After several washes in phosphate-buffered saline (PBS) (Sigma), nonspecific sites were blocked with 3% bovine serum albumin (Sigma) in PBS for 30 minutes. The cells were incubated for 90 minutes at room temperature or overnight at 4° C. simultaneously with the primary antibody of collagen type I, IV, laminin, fibronectin, α-smooth muscle actin, nephrin and synaptopodin. Bound antibodies were visualized using AlexaFluor® 488 chicken anti-rabbit (Invitrogen) and AlexaFluor 555 goat anti-mouse (Invitrogen) 1:400 in PBS for 30 minutes. Post-fixation was done with 2% PFA for 15 minutes. Cell nuclei were counterstained with Hoechst stains (bisBenzimide H 33342 trihydrochloride; Invitrogen) and slides were mounted with Vectashield® mounting media (Vector Laboratories, Inc; Burlingame, Calif.). Images were captured and fluorescence intensity measurements were performed with a Zeis Axio Observer.A1 inverted fluorescence microscope (ZEISS; Oberkochen, Germany) or a Nikon eclipse Ti-E confocal laser scanning microscopy (Nikon).

Reverse transcription-polymerase chain reaction (RT-PCR): Total RNA was extracted from podocytes with the use of RNeasy Plus Mini kit (Qiagen; Valencia, Calif.) or TRlzol reagent (ThermoFisher Scientific), respectively. Reverse transcription was performed using RNA to cDNA EcoDry Premix kit (Takara Bio; Shiga, Japan). The resulting cDNA was amplified by TaqMan probe-based PCR (ThermoFisher Scientific) with a LightCycler 480 instrument (Roche; Basel, Switzerland). The abundance of each target mRNA was normalized by that of glyceraldehyde 3-phosphate dehydrogenase (GAPDH) mRNA. Taqman primer-probes used were as follows: nephrin (Assay ID), synaptopodin (Assay ID), collagen IV (Assay ID), WT1 (Assay ID) and GAPDH (Assay ID).

Statistical analyses: Numerical data is expressed as mean±standard deviation (SD). Analysis was performed using statistical software (Graphpad Prism; La Jolla, Calif.). Statistical significance was accepted at p<0.05. Experiments were performed in triplicate or quadruplicate.

Results

Evaluation of deposited extracellular matrix on decellularized matrix: To acquire maximum ECM deposition after decellularization of human fibroblasts, various decellularization media (0.5% Sodium deoxycholate with or without Triton X-100 and EDTA) and incubation times were assessed. SDS-PAGE and complementary densitometric analysis (FIG. 2) revealed that the maximum (p<0.0001) collagen type I deposition was achieved after decellularization with 0.5% Sodium deoxycholate with Triton X-100 and EDTA for 15 minute incubation. After decellularization the decellularized matrix was stained using immunofluorescence to evaluate deposition of collagen type I, collagen type IV, and fibronectin. Immunofluorescence analysis of decellularized matrix confirmed the deposition of collagen type I, collagen type IV, and fibronectin (FIG. 2).

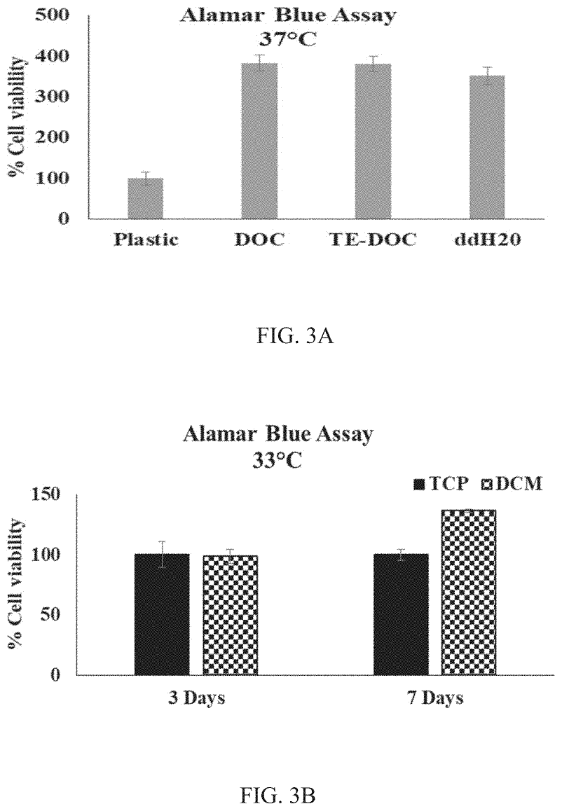

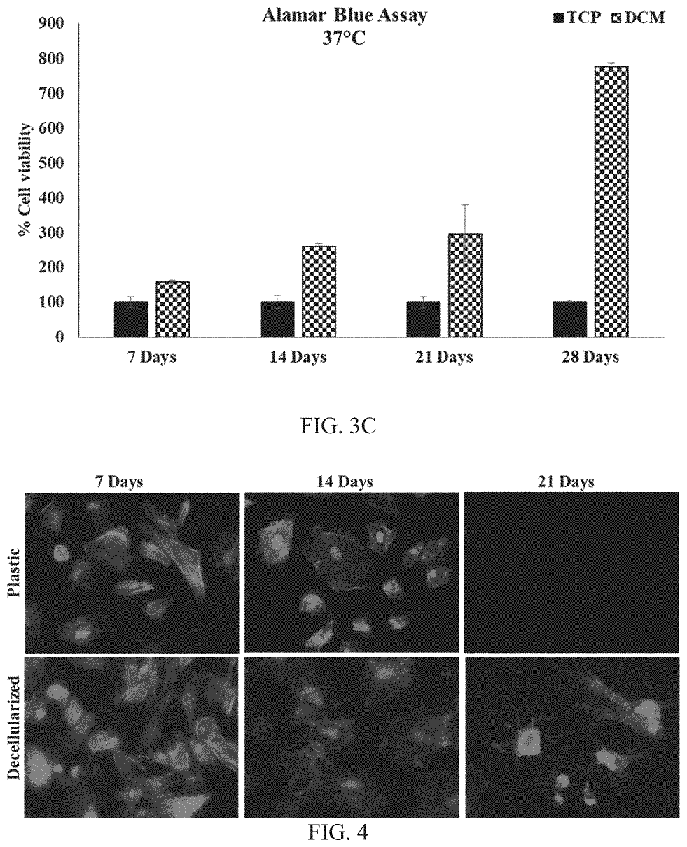

Evaluation of podocytes viability on decellularized matrix: AlamarBlue® cell metabolic activity assay was performed to quantify viability of podocytes on decellularized matrix. Three methods of decellularization were evaluated (i) DOC—0.5% Sodium deoxycholate, (ii) TE-DOC—DOC—0.5% Sodium deoxycholate with Triton X-100 and EDTA and (iii) double distilled water after 21 days of culture at 37° C. AlamarBlue® assay demonstrated that decellularized matrix helped to increase viability of podocytes (FIG. 3A). Moreover significant increase in the viability of podocytes was observed at various culture conditions (proliferation at 33° C. (FIG. 3B) and differentiation at 37° C. (FIG. 3C)) and up to 28 days.

Assessment of podocyte morphology on decellularized matrix: The influence of decellularized matrix on morphology of podocytes was evaluated using actin staining by immunofluorescence. The differentiation of human podocytes on plastic or decellularized fibroblast ECM rich substrate was examined. Podocytes cultured on decellularized matrix showed native morphology with interdigitating foot process up to 21 days under differentiation conditions. However, podocytes cultured on plastic did not show foot process and were not viable up to 21 days (FIG. 4).

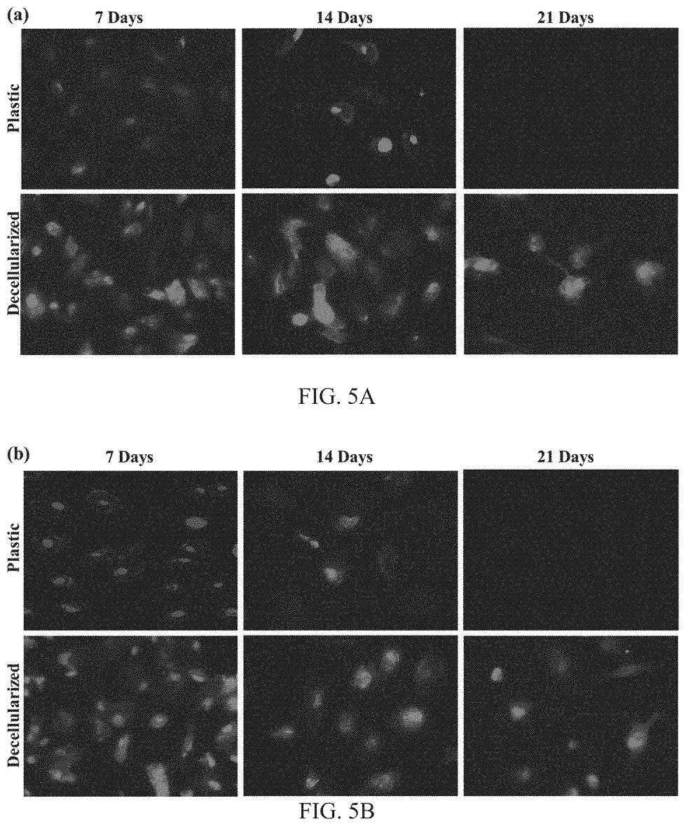

Evaluation of podocyte specific markers: The effect of decellularized matrix on expression of podocytes specific markers were evaluated by western blotting and immunofluorescence staining. Using western blotting, the protein levels of synaptopodin, nephrin, WT1, and actin were evaluated. The podocytes cultured on decellularized ECM rich substrate showed higher expression of synaptopodin (FIG. 5A) and nephrin (FIG. 5B) up to 21 days under differentiation conditions.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All documents cited herein are incorporated by reference.

1. A method of growing podocytes in culture, the method comprising:
a. contacting a tissue culture substrate with cells;
b. growing the cells on the tissue culture substrate;
c. inducing the cells to produce an extracellular matrix (ECM);
d. decellularizing the ECM to produce a decellularized ECM; and
e. contacting the decellularized extracellular matrix with podocytes under conditions suitable to grow the podocytes, wherein the podocytes are grown in culture.

2. The method of claim 1, wherein the cells are selected from the group consisting of fibroblasts, stem cells, glomerular parietal epithelial cells, renal pericytes, mesangial cells, and renal tubule epithelial cells.

3. The method of claim 1, wherein the tissue culture substrate is selected from the group consisting of a tissue culture plastic, a glass container, a bioceramic container, a stainless steel container, and a polymeric container.

4. The method of claim 3, wherein the tissue culture substrate is a tissue culture plastic.

5. The method of claim 1, wherein inducing the cells to produce ECM comprises contacting the cells with an induction agent capable of inducing collagen production from the cells.

6. The method of claim 5, wherein the induction agent is selected from the group consisting of ascorbic acid phosphate, polysaccharide, hyaluronic acid, polyethylene glycol, and polyvinylpyrrolidone.

7. The method of claim 1, wherein decellularizing the ECM to produce a decellularized ECM comprises contacting the cells and ECM with a decellularizing agent capable of reducing or eliminating cellular components from the ECM.

8. The method of claim 7, wherein the decellularizing agent is selected from the group consisting of a mild detergent, an enzyme, double distilled water, acids, bases, and mechanical decellularization.

9. The method of claim 8, wherein the mild detergent is selected from the group consisting of sodium deoxycholate, Triton X-100, CHAPS, and sodium dodecyl sulfate (SDS).

10. The method of claim 1, wherein the method further comprises differentiating the podocytes grown in culture.

11. A podocyte cell produced by the method of claim 1.

12. A pharmaceutical composition comprising a podocyte cell of claim 11 and a pharmaceutically acceptable carrier.

13. A podocyte cell culture system, the podocyte cell culture system comprising:
a. a decellularized extracellular matrix (ECM);
b. a tissue culture substrate; and
c. a podocyte.

14. The podocyte cell culture system of claim 13, wherein the tissue culture substrate is selected from the group consisting of a tissue culture plate, a glass container, a bioceramic container, a stainless steel container, and a polymeric container.

15. The podocyte cell culture system of claim 13, wherein the decellularized ECM is produced by (a) growing cells on a tissue culture substrate; (b) inducing the cells to produce an extracellular matrix (ECM); and (c) decellularizing the ECM to produce a decellularized ECM.

16. A method of determining if a subject with lupus is at risk of developing glomerulonephritis, the method comprising:
a. obtaining sera from a subject with lupus;
b. contacting the sera with the podocyte cell culture system of claim 13; and
c. detecting damage to the podocyte cell culture, wherein detecting damage to the podocyte cell culture indicates that the subject is at risk of developing glomerulonephritis.

17. A method of determining if a subject with a kidney transplant is at risk of rejecting the kidney transplant, the method comprising:
a. obtaining sera from a subject with a kidney transplant;
b. contacting the sera with the podocyte cell culture system of claim 13; and
c. detecting damage to the podocyte cell culture, wherein detecting damage to the podocyte cell culture indicates that the subject is at risk of rejecting the kidney transplant.

18. The method of claim 17, wherein the method further comprises administering a therapeutic to the subject to reduce the risk of rejecting the kidney transplant.

19. The method of claim 18, wherein the therapeutic is a pharmaceutical composition comprising a podocyte cell and a pharmaceutically acceptable carrier, the podocyte cell produced by a method of growing podocytes in culture comprising:
a. contacting a tissue culture substrate with cells;
b. growing the cells on the tissue culture substrate;
c. inducing the cells to produce an extracellular matrix (ECM);
d. decellularizing the ECM to produce a decellularized ECM; and
e. contacting the decellularized ECM with podocytes under conditions suitable to grow the podocytes.

20. A method of determining if a kidney is at risk to cytotoxicity due to a therapeutic agent, the method comprising:
a. contacting the therapeutic agent with the podocyte cell culture system of claim 13; and
b. detecting damage to the podocyte cell culture, wherein detecting damage to the podocyte cell culture indicates that the kidney is at risk to cytotoxicity due to the therapeutic agent.

21. The method of claim 20, wherein the therapeutic agent is selected from an antibiotic, a chemotherapeutic agent, a therapeutic peptide, and a therapeutic small molecule.
